(19) United States
(12) Patent Application Publication
(10) Pub. No.: US 2014/0350528 A1
(43) Pub. Date: Nov. 27, 2014

(54) SYSTEMS AND METHODS FOR DELIVERING A THERAPEUTIC AGENT

(71) Applicant: SpringLeaf Therapeutics, Inc., Boston, MA (US)

(72) Inventors: Yet-Ming CHIANG, Weston, MA (US); Timothy E. CHIN, San Jose, CA (US); Michael J. CIMA, Winchester, MA (US); J. Richard GYORY, Sudbury, MA (US); Malinda TUPPER, Somerville, MA (US)

(73) Assignee: SpringLeaf Therapeutics, Inc., Boston, MA (US)

(21) Appl. No.: 14/215,485

(22) Filed: Mar. 17, 2014

Related U.S. Application Data
(63) Continuation of application No. 13/157,919, filed on Jun. 10, 2011, now abandoned.
(60) Provisional application No. 61/478,837, filed on Apr. 25, 2011.
(60) Provisional application No. 61/353,960, filed on Jun. 11, 2010.
(60) Provisional application No. 61/353,516, filed on Jun. 10, 2010.
(60) Provisional application No. 61/353,520, filed on Jun. 10, 2010.

(51) Int. Cl. A61M 5/142

(52) U.S. Cl. CPC A61M 5/14248
USPC 604/890.1

(57) ABSTRACT

Devices and methods for delivering a fluid to a patient are disclosed herein. In one embodiment, a method includes providing a wearable delivery device that includes an electrochemical actuator and a reservoir containing a fluid with viscosity greater than 15 cP. The electrochemical actuator can be actuated such that the actuator exerts sufficient force on the reservoir to cause the fluid within the reservoir to be communicated to the patient's body over a time period. In some embodiments, the time period is two hours. In one embodiment, a delivery system includes a reservoir containing a fluid having a viscosity greater than 15 cP and a fluid communicator in fluid communication with the reservoir. An electrochemical actuator is coupled to the reservoir and configured to exert a sufficient force on the reservoir for a time period upon actuation such that the fluid within the reservoir is communicated through the fluid communicator.

### CROSS-REFERENCE TO RELATED APPLICATIONS

This application claims priority to and the benefit of U.S. Provisional Application Ser. No. 61/353,516, filed Jun. 10, 2010, entitled “Systems and Methods for Delivering a Therapeutic Agent”, U.S. Provisional Application Ser. No. 61/353,520, filed Jun. 10, 2010, entitled “Systems and Methods for Delivering a Therapeutic Agent”, U.S. Provisional Application Ser. No. 61/353,960, filed Jun. 11, 2010, entitled “Systems and Methods for Delivering a Therapeutic Agent”, and U.S. Provisional Application Ser. No. 61/478,837, filed Apr. 25, 2011, entitled “Systems and Methods for Delivering a Therapeutic Agent”, the disclosures of which are hereby incorporated by reference in their entirety.

### BACKGROUND

The invention relates generally to medical devices and procedures, including, for example, medical devices and methods for delivering a therapeutic agent to a patient.

Drug delivery involves delivering a drug or other therapeutic compound into the body. Typically, the drug is delivered via a technology that is carefully selected based on a number of factors. These factors can include, but are not limited to, the characteristics of the drug, such as drug dose, pharmacokinetics, complexity, cost, and absorption, the characteristics of the desired drug delivery profile (such as uniform, non-uniform, or patient-controlled), the characteristics of the administration mode (such as the ease, cost, complexity, and effectiveness of the administration mode for the patient, physician, nurse, or other caregiver), or other factors or combinations of these factors.

Conventional drug delivery technologies present various challenges. Oral administration of a dosage form is a relatively simple delivery mode, but some drugs may not achieve the desired bioavailability and/or may cause undesirable side effects if administered orally. Further, the delay from time of administration to time of efficacy associated with oral delivery may be undesirable depending on the therapeutic need. While parenteral administration by injection may avoid seine of the problems associated with oral administration, such as providing relatively quick delivery of the drug to the desired location, conventional injections may be inconvenient, difficult to self-administer, and painful or unpleasant for the patient. Furthermore, injection may not be suitable for achieving certain delivery/release profiles, particularly over a sustained period of time.

Passive transdermal technology, such as a conventional transdermal patch, may be relatively convenient for the user and may permit relatively uniform drug release over time. However, some drugs, such as highly charged or polar drugs, peptides, proteins and other large molecule active agents, may not penetrate the stratum corneum for effective delivery. Furthermore, a relatively long start-up time may be required before the drug takes effect. Thereafter, the drug release may be relatively continuous, which may be undesirable in some cases. Also, a substantial portion of the drug payload may be undeliverable and may remain in the patch once the patch is removed.

Active transdermal systems, including iontophoresis, sonophoresis, and poration technology, may be expensive and may yield unpredictable results. Only some drug formulations, such as aqueous stable compounds, may be suited for active transdermal delivery. Further, modulating or controlling the delivery of drugs using such systems may not be possible without using complex systems.

Some infusion pump systems may be large and may require tubing between the pump and the infusion set, which can impact the quality of life of the patient. Further, infusion pumps may be expensive and may not be disposable. From the above, it would be desirable to provide new and improved drug delivery systems and methods that overcome some or all of these and other drawbacks.

### SUMMARY OF THE INVENTION

Devices and methods for delivering a fluid to a patient are disclosed herein. In one embodiment, a method includes providing a wearable delivery device that includes an electrochemical actuator and a reservoir containing a fluid with viscosity of greater than 15 cP. The electrochemical actuator can be actuated such that the electrochemical actuator exerts sufficient force on the reservoir to cause the fluid within the reservoir to be communicated or delivered to the patient's body over a time period. In one embodiment, a delivery system includes a reservoir containing a fluid having a viscosity greater than 15 cP and a fluid communicator in fluid communication with the reservoir. An electrochemical actuator is coupled to the reservoir and configured to exert a sufficient force on the reservoir for a time period upon actuation such that the fluid within the reservoir is communicated through the fluid communicator. In some embodiments, a transfer structure is disposed between the electrochemical actuator and the reservoir. The transfer structure can be configured to contact the reservoir and transfer the force exerted by the electrochemical actuator to the reservoir upon actuation of the electrochemical actuator.

### BRIEF DESCRIPTION OF THE DRAWINGS

FIG. 1 is a schematic illustration of a delivery system according to an embodiment.

FIG. 2A is a schematic illustration of a portion of a delivery system according to an embodiment illustrating an electrochemical actuator in a charged state; and FIG. 2B is a schematic illustration of the portion of the delivery system of FIG. 2A illustrating the electrochemical actuator as it discharges.

FIG. 3A is a schematic illustration of a portion of a delivery system according to an embodiment illustrating an electrochemical actuator in a charged state and FIG. 3B is a schematic illustration of the portion of the delivery system of FIG. 3A illustrating the electrochemical actuator as it discharges.

FIG. 4A is a perspective view of a delivery system according to an embodiment and FIG. 4B is an exploded view of the delivery system of FIG. 4A.

FIG. 5A is a graph of example doses versus time that can be delivered using a pump with an electrochemical actuator according to an embodiment; and FIG. 5B is a graph of example flow rates versus viscosity.

FIG. 6 is a table showing dimensions for standard gauge needles.

FIG. 7A shows a graph with examples of the calculated effect of viscosity on the required force needed to pump a fluid volume of 10 ml at various rates, and the interrelationship between the diameter of the tube the fluid is being pumped through and the desired rate the fluid is being pumped; and FIG. 7B shows graphs of examples of the force required to dispense various amounts of fluids having various viscosities over time.

### DETAILED DESCRIPTION

Devices, systems and methods are described herein that are configured for use in the delivery of therapeutic agents to a patient's body. Such therapeutic agents can be, for example, one or more drugs and can be in fluid form of various viscosities. In some embodiments, the devices and methods can include a pump device that includes an actuator, such as, for example, an electrochemical actuator, which can have characteristics of both a battery and a pump. Specifically, an electrochemical actuator can include an electrochemical cell that produces a pumping force as the cell discharges without a separate power supply. Thus, the pump device can have relatively fewer parts than a conventional drug pump, such that the pump device is relatively more compact, lightweight, disposable, and reliable than conventional drug pumps. Such drug delivery devices are desirable, for example, for use in delivery devices that are designed to be attached to a patient's body (e.g., a wearable device) or belt, or worn in a holster that can be attached to a patient's body or clothing. These attributes of the pump device may reduce the cost and the discomfort associated with infusion drug therapy.

In some embodiments, such a pump device can be operated with, for example, a controller and/or other circuitry, operative to regulate drug or fluid flow from the pump device. Such a controller may permit implementing one or more release profiles using the pump device, including release profiles that require uniform flow, non-uniform flow, continuous flow, discontinuous flow, programmed flow, scheduled flow, user-initiated flow, or feedback responsive flow, among others. In other embodiments, the delivery rate of the pump device can be controlled by electrical circuitry configured to control the discharge rate of the actuator. Thus, the pump device may effectively deliver a wider variety of drug therapies than other pump devices.

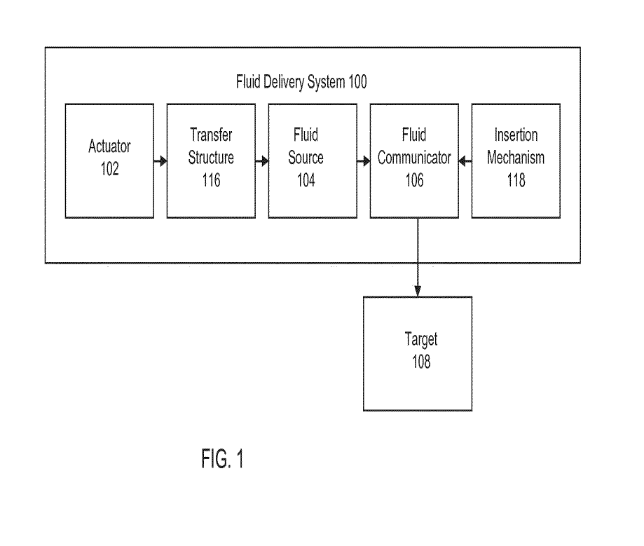

FIG. 1 is a schematic block diagram illustrating an embodiment of a fluid delivery system 100 (also referred to herein as “delivery device” or “drug delivery device”). The fluid delivery system 100 includes an electrochemical actuator 102, a fluid source 104 and a fluid communicator 106. The system 100 also includes an insertion mechanism 118 and an optional transfer structure 116. The fluid source 104 can contain a fluid (i.e., a therapeutic agent) to be delivered into a target 108 via the fluid communicator 106. The target 108 can be, for example, a human or other mammalian body in need of a drug therapy or prophylaxis.

The electrochemical actuator 102 can actuate or otherwise create a pumping force to deliver the fluid from the fluid source 104 into the fluid communicator 106 as described in more detail below. In some embodiments, the electrochemical actuator 102 can be a device that experiences a change in volume or position in response to an electrochemical reaction that occurs therein. For example, the electrochemical actuator 102 can include a charged electrochemical cell, and at least a portion of the electrochemical cell can actuate as the electrochemical cell discharges. Thus, the electrochemical actuator 102 can be considered a self-powered actuator or a combination battery and actuator.

The fluid source 104 can be a reservoir, pouch, bag, chamber, barrel, bladder, or other known device that can contain a drug in fluid form therein. The fluid communicator 106 can be in, or can be moved into, fluid communication with the fluid source 104. The fluid communicator 106 can be, for example, a needle, catheter, cannula, infusion set, or other known drug delivery conduit that can be inserted into or otherwise associated with the target body for drug delivery.

In some embodiments, the fluid source 104 can be any component capable of retaining a fluid or drug in fluid form. In some embodiments, the fluid source 104 may be disposable (e.g., not intended to be refillable or reusable). In other embodiments, the fluid source 104 can be refilled, which may permit reusing at least a portion of the device and/or varying the drug or fluid delivered by the device. In some embodiments, the fluid source 104 can be sized to correlate with the electrochemical potential of the electrochemical actuator 102. For example, the size and/or volume of the fluid source 104 can be selected so that the fluid source 104 becomes about substantially empty at about the same time that the electrochemical actuator 102 becomes about substantially discharged. By optimizing the size of the fluid source 104 and the amount of drug contained therein to correspond to the driving potential of the electrochemical actuator 102, the size and/or cost of the device may be reduced. In other embodiments, the electrochemical actuator 102 may be oversized with reference to the fluid source 104. In some embodiments, the delivery system 100 can include more than one fluid source 104. Such a configuration may permit using a single device to deliver two or more drugs or fluids. The two or more drugs or fluids can be delivered discretely, simultaneously, alternating, according to a program or schedule, or in any other suitable manner. In such embodiments, the fluid sources 104 may be associated with the same or different electrochemical actuators 102, the same or different fluid communicators 106, the same or different operational electronics, or the same or different portions of other components of the delivery system.

In some embodiments, the electrochemical actuator 102 applies force directly to the fluid source 104 such that the fluid within the fluid source 104 is expelled out of the fluid source and through the fluid communicator 106. In some embodiments, the delivery device 100 includes a transfer structure 116 disposed between the fluid source 104 and the electrochemical actuator 102. The transfer structure 116 includes a surface configured to contact the fluid source 104 upon actuation of the actuator 102 such that a force exerted by the electrochemical actuator 102 is transferred from the transfer structure 116 to the fluid source 104. The transfer structure 116 can include one or more components. For example, the transfer structure 116 can be a single component having a surface configured to contact the fluid source 104. In some embodiments, the transfer structure 116 can include one or more members having a surface configured to contact the fluid source 104 upon activation of the electrochemical actuator 102. In some embodiments, the transfer structure 116 is a substantially planar or flat plate.

The insertion mechanism 118 can be used to insert the fluid communicator 106 into the target 108 (e.g., a desired injection site on the patient's body). The insertion mechanism can include one or more energy storage mechanisms such as a spring. For example, a variety of different types of springs can be used, such as, compression, extension, spring washers, Belleville, tapered, or other types of springs to achieve a desired output. The insertion mechanism 118 can include a penetration cannula having one end configured to penetrate the patient's skin and another end configured to puncture the fluid source 104. The penetration cannula can definite a lumen and be movably disposed within a lumen of the fluid communicator 106.

In some embodiments, the fluid delivery system 100 can be used to deliver a drug formulation which comprises as drug, including an active pharmaceutical ingredient. In other embodiments, the fluid delivery system 100 may deliver a fluid that does not contain a drug. For example, the fluid may be a saline solution or a diagnostic agent, such as a contrast agent. Drug delivery can be subcutaneous, intravenous, intraarterial, intramuscular, intracardiac, intraosseous, intradermal, intrathecal, intraperitoneal, intratumoral, intratympnic, intraaural, topical, epidural, and/or peri-neural depending on, for example, the location of the fluid communicator 106 and/or the entry location of the drug.

The drug (also referred to herein as “a therapeutic agent” or “a prophylactic agent”) can be in a pure form or formulated in a solution, a suspension, or an emulsion, among others, using one or more pharmaceutically acceptable excipients known in the art. For example, a pharmaceutically acceptable vehicle for the drug can be provided, which can be any aqueous or non-aqueous vehicle known in the art. Examples of aqueous vehicles include physiological saline solutions, solutions of sugars such as dextrose or mannitol, and pharmaceutically acceptable buffered solutions, and examples of non-aqueous vehicles include fixed vegetable oils, glycerin, polyethylene glycols, alcohols, and ethyl oleate. The vehicle may further include antibacterial preservatives, antioxidants, tonicity agents, buffers, stabilizers, or other components.

Although the fluid delivery system 100 and other systems and methods described herein are generally described as communicating drugs into a human body, such systems and methods may be employed to deliver any fluid of any suitable biocompatibility or viscosity into any object, living or inanimate. For example, the systems and methods may be employed to deliver other biocompatible fluids into living beings, including human beings and other animals. Further, the systems and methods may deliver drugs or other fluids into living organisms other than human beings, such as animals and plant life. Also, the systems and methods may deliver any fluids into any target, living or inanimate.

The systems and methods described herein are generally systems and methods of delivering fluids using a delivery device 100 that includes an electrochemical actuator 102, such as a self-powered actuator and/or combined battery and actuator. Example embodiments of such electrochemical actuators are generally described in U.S. Pat. No. 7,541,715, entitled “Electrochemical Methods, Devices, and Structures” by Chiang et al., U.S. Patent Pub. No. 2008/0257718, entitled “Electrochemical Actuator” by Chiang et al., and U.S. Patent Pub. No 2009/0014320, entitled “Electrochemical Actuator” by Chiang et al., and U.S. Pat. No. 7,828,771, entitled “Systems and Methods for Delivering Drugs” by Chiang et al., (“the '771 Patent”), the disclosures of each of which is incorporated herein by reference. Such electrochemical actuators can include at least one component that responds to the application of a voltage or current by experiencing a change in volume or position. The change in volume or position can produce mechanical work that can then act on a fluid source (e.g., fluid source 104) or may be transferred to a fluid source, such that a fluid can be delivered out of the fluid source.

In some embodiments, the electrochemical actuator 102 can include a positive electrode and a negative electrode, at least one of which is an actuating electrode. These and other components of the electrochemical actuator can form an electrochemical cell, which can in Some embodiments initially be charged. For example, the electrochemical cell may begin discharging when a circuit between the electrodes is closed, causing the actuating electrode to actuate. The actuating electrode can thereby perform work upon another structure, such as the fluid source, or a transfer structure associated with the fluid source, as described in more detail below. The work can then cause fluid to be pumped or otherwise dispensed from the fluid source into the target 108.

More specifically, the actuating electrode of the electrochemical actuator 102 can experience a change in volume or position when the closed circuit is formed, and this change in volume or position can perform work upon the fluid source or transferring structure. For example, the actuating electrode may expand, bend, buckle, fold, cup, elongate, contract, or otherwise experience a change in volume, size, shape, orientation, arrangement, or location, such that at least a portion of the actuating electrode experiences a change in volume or position. In some embodiments, the change in volume or position may be experienced by a portion of the actuating electrode, while the actuating electrode as a whole may experience a contrary change or no change whatsoever. It is noted that the delivery device 100 cart include more than one electrochemical actuator 102. For example, in some embodiments, the delivery device 100 can include one or more electrochemical actuators 102 arranged in series, parallel, or some combination thereof. In some embodiments, a number of such electrochemical actuators 102 may be stacked together. As another example, concurrent or sequenced delivery of multiple agents can be achieved by including one or more electrochemical actuators 102 acting on two or more fluid sources.

The delivery system 100 can also include a housing (not shown in FIG. 1) that can be removably or releasably attached to the skin of the patient. The various components of the delivery system 100 can be fixedly or releasably coupled to the housing. To adhere the delivery device 100 to the skin of a patient, a releasable adhesive can at least partially coat an underside of the housing. The adhesive can be non-toxic, biocompatible, and releasable from human skin. To protect the adhesive until the device is ready for use, a removable protective covering can cover the adhesive, in which case the covering can be removed before the device is applied to the skin. Alternatively, the adhesive can be heat or pressure sensitive, in which case the adhesive can be activated once the device is applied to the skin. Example adhesives include, but are not limited to, acrylate based medical adhesives of the type commonly used to affix medical devices such as bandages to skin. However, the adhesive is not necessary, and may be omitted, in which case the housing can be associated with the skin, or generally with the body, in any other manner. For example, a strap or band can be used.

The housing can be formed from a material that is relatively lightweight and flexible, yet sturdy. The housing also can be formed from a combination of materials such as to provide specific portions that are rigid and specific portions that are flexible. Example materials include plastic and rubber materials, such as polystyrene, polybutene, carbonate, urethane rubbers, butene rubbers, silicone, and other comparable materials and mixtures thereof, or a combination of these materials or any other suitable material can be used.

In some embodiments, the housing can include a single component or multiple components. In some embodiments, the housing can include two portions: a base portion and a movable portion. The base portion can be suited for attaching to the skin. For example, the base portion can be relatively flexible. An adhesive can be deposited on an underside of the base portion, which can be relatively flat or shaped to conform to the shape of a particular body part or area. In some embodiments, the base portion can include the fluid communicator 106 and the insertion mechanism 118, while the movable portion includes the fluid source 104. The movable portion can be sized and shaped for association with the base portion. In wane embodiments, the two portions can be designed to lock together, such as via a locking mechanism. In some cases, the two portions can releasably lock together, such as via a releasable locking mechanism, so that the movable portion can be removably associated with the base portion. To assemble such a housing, the movable portion can be movable with reference to the base portion between an unassembled position and an assembled position. In the assembled position, the two portions can form a device having an outer shape suited for concealing the device under clothing. Various example embodiments of a housing are described in the '771 Patent.

The size, shape, and weight of the delivery device 100 can, be selected so that the delivery device 100 can be comfortably worn on the skin after the device is applied via the adhesive. For example, the delivery device 100 can have a size, for example, in the range of about 1.0″×1.0″×0.1″ to about 5.0″×5.0″×1.0″, and in seine embodiments in a range of about 2.0″×2.0″×0.25″ to about 4.0″×4.0″×0.67″. The weight of the delivery device 100 can be, for example, in the range of about 5 g to about 200 g, and in some embodiments in a range of about 15 g to about 100 g. The delivery device 100 can be configured to dispense a volume in the range of about 0.1 ml to about 1,000 ml. In some embodiments, the delivery device is, capable of delivering biologics having concentrations in any sub-range within this range. For example, in some embodiments, the pump device is capable of delivering a volume in a range of about 0.3 ml to about 500 ml. In some embodiments, the pump device is capable of delivering a volume in a range of about 0.5 ml to about 100 ml. In some embodiments, the pump device is capable of delivering a volume in a range of about 0.1 ml to about 20 ml, and in some cases in the range of about 0.5 ml to about 20 ml, such as between about 5 ml and about 10 ml. The shape of the delivery device 100 can be selected so that the delivery device 100 can be relatively imperceptible under clothing. For example, the housing can be relatively smooth and free from sharp edges. However, other sizes, shapes, and/or weights are possible.

As mentioned above, an electrochemical actuator 102 can be used to cause the fluid delivery device 100 to deliver a drug-containing or non-drug containing fluid into a human patient or other target 108. Such a fluid, delivery system 100 can be embodied in a relatively small, self-contained, in part, and disposable device, such as a patch device that can be removably attached to the skin of patient as described above. The delivery device 100 can be relatively small and self-contained because the electrochemical actuator 102 serves as both the battery and a pump. The small and self-contained nature of the delivery device 100 advantageously may permit concealing the device beneath clothing and may allow the patient to continue normal activity as the drug is delivered. Unlike conventional drug pumps, external tubing to communicate fluid from the fluid reservoir into the body can be minimized or even eliminated in order to maximize the volume of fluid delivered from the reservoir to the body, thus reducing waste. Such tubing can instead be contained within the delivery device, and a needle or other fluid communicator can extend from the device into the body.

The electrochemical actuator 102 can initially be charged, and can begin discharging once the delivery device 100 is activated to pump or otherwise deliver the drug or other fluid into the target 108. Once the electrochemical actuator 102 has completely discharged or the fluid source 104 (e.g. reservoir) is empty, the delivery device 100 can be removed. The small and inexpensive nature of the electrochemical actuator. 102 and other components of the device may permit disposing of the entire delivery device 100 after a single use. The delivery device 100 can permit drug delivery, such as subcutaneous or intravenous drug delivery, over a time period that can vary from several minutes to several days. In some embodiments, the delivery device 100 is capable of delivering a drug in any stab-range within this range. For example, in some embodiments, the delivery device 100 is capable of delivering a drug in a range of about 15 minutes to about 7 days. In some embodiments, the delivery device 100 is capable of delivering a drug in a range of about 30 minutes to about 72 hours. In some embodiments, the delivery device 100 is capable of delivering a drug in a range of about 30 minutes to about 24 hours. Subsequently, the delivery device 100 can be removed from the body and discarded.

The fluid delivery device can deliver a fluid at a relatively uniform (non-pulsative) rate over a wide range of time periods such as, for example, ranging from several minutes up to several days. The actuator can be configured to linearly displace (either volumetrically or by bending) up to about 1 mm per hour. As described above, a controller and/or other electrical circuitry can be used to regulate fluid flow from the pump device or the positive and negative electrodes can be designed to bend or displace at a rate slower than 1 mm per hour. Other components of the pump device such as, for example, the size and shape of the fluid reservoir can be designed to provide optimal flow rates and delivery volumes.

The ability to customize the delivery profile and deliver therapies over long periods of time combined with the wearable nature (whether patch or holster) of the fluid delivery device provides many benefits over syringe-based delivery systems. For example, it is impractical to expect a patient to sit in a doctor's office or clinic while a bench top syringe-based system delivers a therapy to the patient. Furthermore, syringe bases delivery systems require extra tubing to communicate the fluid from the syringe to the patient. This extra tubing is either oversized diameter to reduce the force required to pump the fluid, thus wasting a lot of the drug trapped in the tubing, or smaller diameter to reduce the wasted drug, thus increasing the force required to deliver the drug. Since the amount of force capable of being generated by a syringe-based pump is limited, the delivery time often needs to be extended.

In use, the delivery device 100 can be placed in contact with the target 108 (e.g. placed on the surface of a patient's body), such that the fluid communicator 106 (e.g., as needle, cannula, etc.) is disposed adjacent to a desired injection site. The insertion mechanism 118 can be used to insert the fluid communicator 106 into the patient's body at the target 108 as described in more detail below with reference to specific embodiments. In some embodiments, a separate insertion device can be used as described in the '771 Patent. In some embodiments, the fluid communicator 106 can be actuated simultaneously with the actuation of the electrochemical actuator 102. With the fluid communicator 106 inserted into the target 108, the electrochemical actuator 102 can then be actuated to apply a force on the fluid source 104, causing the fluid to be delivered through the fluid communicator 106 and into the target 108. For example, as the electrochemical actuator 102 is actuated, the actuator 102 will be displaced and will contact and apply a force to the fluid source 104 to pump the fluid out of the fluid source 104, through the fluid communicator 106, and into the target 108. If the delivery device 100 includes a transfer structure 116 disposed between the fluid source 104 and the electrochemical actuator 102, the electrochemical actuator 102 will contact the transfer structure 116 and that force will in turn be transferred to the fluid source 104 to pump the fluid out of the fluid source 104.

Having described above various general principles, several exemplary embodiments of these concepts are now described. These embodiments are only examples, and many other configurations of a delivery system and/or the various components of a delivery system, are contemplated. For example, in some embodiments, the delivery device 100 can include an amplification mechanism coupled to the electrochemical actuator configured to increase at least one of the force, displacement, or the time period the force is exerted by the actuator on the reservoir. Example embodiments of such amplification mechanisms are generally described in U.S. application Ser. No. 13/102,657, filed May 6, 2011, entitled “Systems And Methods For Delivering a Therapeutic Agent Using Mechanical Advantage.” In other embodiments, the delivery device 100 can include an electrochemical actuator configured as an elongate plate, which bends when actuated. The actuator can be clamped or otherwise constrained at one end, so that the actuator is cantilevered from that end. Example embodiments of such an electrochemical actuator having a clamped end are generally described in U.S. application Ser. No. 13/102,695, filed May 6, 2011, entitled “Systems And Methods For Delivering a Therapeutic Agent.” In still other embodiments, the delivery device can include multiple electrochemical actuators, multiple mechanical actuators, and/or combinations thereof. Example embodiments of such delivery devices having multiple electrochemical and/or mechanical actuators are generally described in U.S. application Ser. No. 13/101,749, filed May 5, 2011, entitled “Systems And Methods For Delivering a Therapeutic Agent,” and U.S. application Ser. No. 13/101,798 filed May 5, 2011, entitled “Systems And Methods For Delivering a Therapeutic Agent Using Mechanical Advantage,” The disclosures of each of the applications referenced above are incorporated herein by reference.

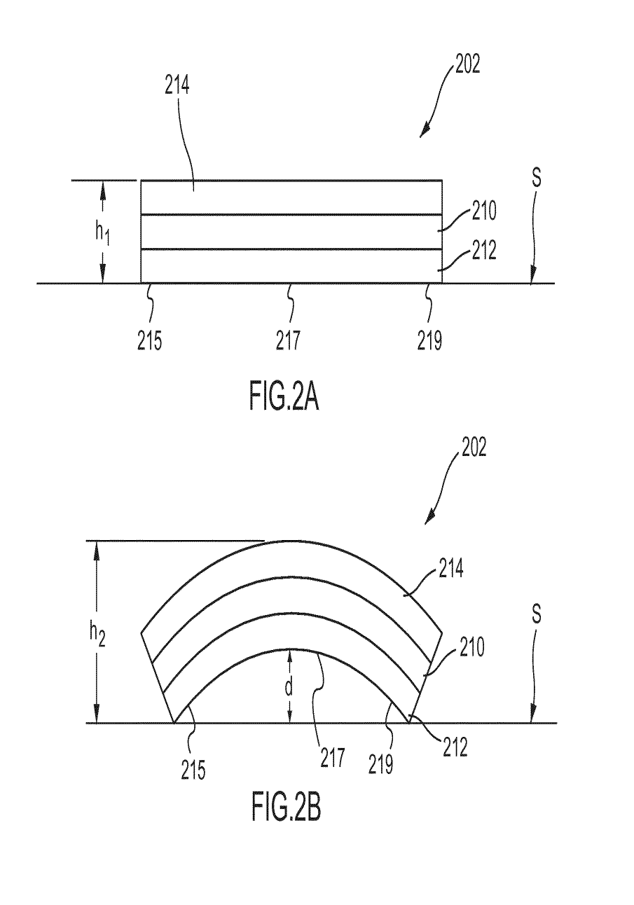

FIGS. 2A and 2B are schematic illustrations of an embodiment of an electrochemical actuator 202 that can be used in a delivery device as described herein. As shown, in this embodiment, the electrochemical actuator 202 can include a positive electrode 210, a negative electrode 212, and an electrolyte 214. These components can form an electrochemical cell that can initially be discharged and then charged before use, or can be initially charged, as shown in FIG. 2A. The positive electrode 210 can be configured to expand or displace in the presence of the electrolyte 214. When a circuit between the electrodes 210, 212 is closed, current can travel from the positive electrode 210 to the negative electrode 212. The positive electrode 210 can then experience a change in volume or shape, resulting in longitudinal displacement of at least a portion of the positive electrode 210, as shown in FIG. 2B. For example, the actuator 202 can have an overall height h1 when it is charged (prior to actuation), as shown in FIG. 2A, and an overall height of h2 when it is discharged or actuated, such that the actuator 202 has a displacement or stroke that is equal to h2-h1. Said another way, the actuator 202 can have a first end portion 215, a second end portion 219 and a medial portion 217 disposed between the first end portion 215 and the second end portion 219. The actuator prior to actuation (prior to discharge) can be supported on a surface S of the delivery device in which the actuator 202 is disposed, and when the actuator 202 is discharged at least the medial portion 217 can displace (e.g., bend or flex) a non-zero distance d from the surface S. The stroke of the actuator 202 can be substantially equal to that non-zero distance d. As the actuator 202 is displaced, the actuator 202 can exert a pumping force or pressure on a fluid reservoir (not shown) and/or an associated transfer structure (not shown) coupled thereto. The pumping force or pressure exerted by the actuator 202 can cause a volume of fluid (e.g., a therapeutic agent) to be pumped out of the fluid reservoir. Thus, the electrochemical actuator 202 can be considered a self-powered electrochemical pump.

In this embodiment, the electrochemical actuator 202 has a positive electrode 210 selected to have a lower chemical potential for the working ion when the electrochemical actuator 202 is charged, and is thereby able to spontaneously accept working ions from the negative electrode 212 as the actuator is discharged. In some embodiments, the working ion can include, but is not limited to, the proton or lithium ion. When the working ion is lithium, the positive electrode 210 can include one or more lithium metal oxides including, for example, LiCoO₂, LiFePO₄, LiNiO₂, LiMn₂O₄, LiMnO₂, LiMnPO₄, Li₄Ti₅O₁₂, and their modified compositions and solid solutions; oxide compound comprising one or more of titanium oxide, manganese oxide, vanadium oxide, tin oxide, antimony oxide, cobalt oxide, nickel oxide or iron oxide; metal sulfides comprising one or more of TiSi₂, MoSi₂, WSi₂, and their modified compositions and solid solutions; a metal, metal alloy, or intermetallic compound comprising one or more of aluminum, silver, gold, boron, bismuth, gallium, germanium, indium, lead, antimony, silicon, tin, or zinc; a lithium-metal alloy; or carbon comprising one or more of graphite, a carbon fiber structure, a glassy carbon structure, a highly oriented pyrolytic graphite, or a disordered carbon structure. The negative electrode 212 can include, for example, lithium metal, a lithium metal alloy, or any of the preceding compounds listed as positive electrode compounds, provided that such compounds when used as a negative electrode are paired with a positive electrode that is able to spontaneously accept lithium from the negative electrode when the actuator is charged. These are just some examples, as other configurations are also possible.

In some embodiments, the electrochemical actuator can include an anode, a cathode, and a species, such as a lithium ion. In some embodiments, a source of lithium ion is the electrolyte which is made up an organic solvent such as PC, propylene carbonate, GBL, gamma butyl lactone, dioxylane, and others, and an added electrolyte. Some example electrolytes include LiPF6, LiBr, LiBF4. At least one of the electrodes can be an actuating electrode that includes a first portion and a second portion. The portions can have at least one differing characteristic, such that in the presence of a voltage or current, the first portion responds to the species in a different manner than the second portion. For example, the portions can be formed from different materials, or the portions can differ in thickness, dimension, porosity, density, or surface structure, among others. The electrodes can be charged, and when the circuit is closed, current can travel. The species can, intercalate, de-intercalate, alloy with, oxide, reduce, or Plate with the first portion to a different extent than the second portion. Due to the first portion responding differently to the species than the second portion, the actuating electrode can experience a change in one or more dimensions, volume, shape, orientation, or position.

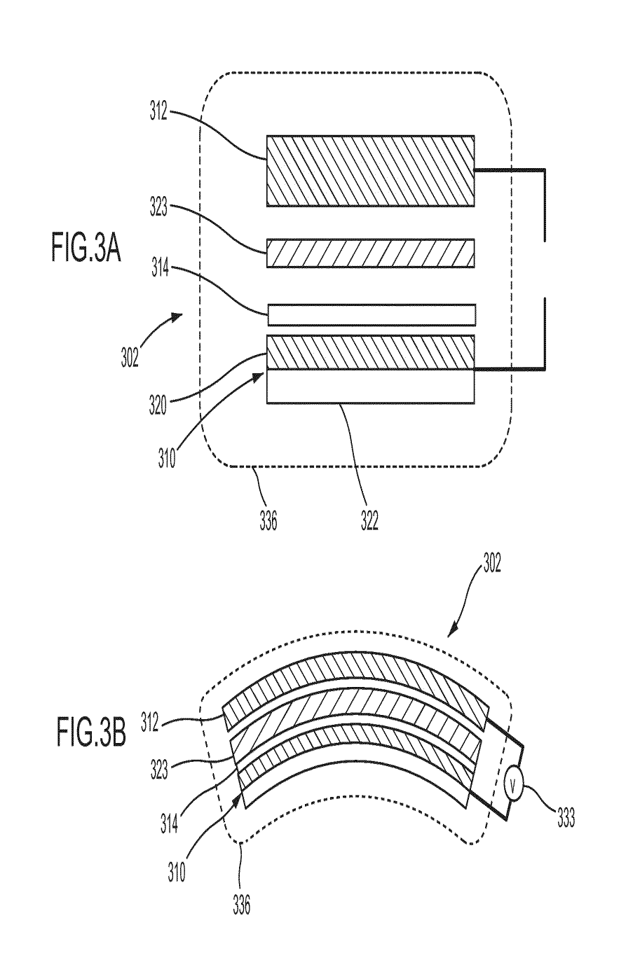

Another example of an electrochemical actuator is shown in the embodiment illustrated in FIGS. 3A and 3B. As shown in FIG. 3A, an electrochemical actuator 302 can include a negative electrode 312 in electrical communication with a positive electrode 310 collectively forming an electrochemical cell. Positive electrode 310 may include a first portion 320 and a second portion 322. In some embodiments, first portion 320 and second portion 322 are formed of different materials. Portions 320 and 322 may also have different electrical potentials. For example, first portion 320 may include a material that can intercalate, de-intercalate, alloy with, oxidize, reduce, or plate a species to a different extent than second portion 322. Second portion 322 may be formed of a material that does not substantially intercalate, de-intercalate, or alloy with, oxidize, reduce, or plate the species. In some embodiments, first portion 320 may be formed of a material including one or more of aluminum, antimony, bismuth, carbon, gallium, silicon, silver, tin, zinc, or other materials which can expand upon intercalation or alloying or compound formation with lithium. In one embodiment, first portion 320 is formed with aluminum, which can expand upon intercalation with lithium. Second portion 322 may be formed of copper, since copper does not substantially intercalate or alloy with lithium. In some instances, second portion 322 may act as a positive electrode current collector, and may extend outside the electrochemical cell, e.g., to form a tab or current lead. In other embodiments, second portion 322 may be joined to a tab or current lead that extends outside the cell. Negative electrode 312 may also include a current collector. Electrochemical actuator 302 may include a separator 323. The separator 323 may be, for example, a porous separator film, such as a glass fiber cloth, or a porous polymer separator. Other types of separators, such as those used in the construction of lithium ion batteries, may also be used. The electrochemical actuator 302 may also include an electrolyte 314, which may be in the form of a liquid, solid, or a gel. The electrolyte may contain an electrochemically active species, such as that used to form the negative electrode. Electrochemical actuator 302 may also include an enclosure 336, such as a polymer packaging, in which negative electrode 312, positive electrode 310 and separator 323 can be disposed.

As illustrated in FIG. 3B, the electrochemical cell may have a voltage 333, such that, when a closed circuit is formed between the negative electrode 312 and the positive electrode 310, an electric current may flow between the negative electrode 312 and the positive electrode 310 through the external circuit. If negative electrode 312 is a lithium metal electrode and the electrolyte contains lithium ions, lithium ion current can flow internally from the negative electrode 312 to the positive electrode 310. The intercalation of first portion 320 with lithium can result in a dimensional change, such as a volume expansion. In some instances, this volume expansion may reach at least 25%, at least 50%, at least 75%, at least 100%, at least 150%, at least 200%, at least 250%, or at least 300% compared to the initial volume. High volume expansion may occur, for example, when first portion 320 is saturated with lithium. As first portion 320 increases in volume due to intercalation of lithium, second portion 322 to which first portion 320 may be bonded, may not substantially expand due to minimal or no intercalation of lithium. First portion 320 thus provides a mechanical constraint. This differential strain between the two portions causes positive electrode 310 to undergo bending or flexure. As a result of the dimensional change and displacement of the positive electrode 310, electrochemical actuator 302 can be displaced from as first orientation to a second orientation. This displacement can occur whether the volumetric or dimensional change (e.g., net volume change) of the electrochemical cell due to the loss of lithium metal from the negative electrode 312 and formation of lithium intercalated compound or lithium alloy at the positive electrode 310, is positive, zero, or negative. In some cases, the actuator displacement may occur with a volumetric or dimensional change (e.g., net volume change) of the electrochemical actuator 302, or portion thereof that is positive. In some cases, the actuator displacement may occur with a volumetric or dimensional change (e.g., net volume change) of the electrochemical actuator 302, or portion thereof that is zero. In some cases, the actuator displacement may occur with a volumetric or dimensional change (e.g., net volume change) of the electrochemical actuator 302, or portion thereof that is negative.

As used herein, “differential strain” between two portions can refer to the difference in response (e.g., actuation) of each individual portion upon application of a voltage or current to the two portions. That is, a system as described herein may include a component including a first portion and a second portion associated with (e.g., may contact, may be integrally connected to) the first portion, wherein, under essentially identical conditions, the first portion may undergo a volumetric or dimensional change and the second portion does not undergo volumetric or dimensional change, producing strain between the first and second portions. The differential strain may cause the component, or a portion thereof, to be displaced from a first orientation to a second orientation. In some embodiments, the differential strain may be produced by differential intercalation, de-intercalation, alloying, oxidation, reduction, or plating of a species with one or more portions of the actuator system.

For example, the differential intercalation, de-intercalation, alloying, oxidation, reduction, or plating of first portion 320 relative to second portion 322 can be accomplished through several means. La one embodiment, first portion 320 may be formed of a different material than second portion 322, wherein one of the materials substantially intercalates, de-intercalates, alloys with, oxidizes, reduces, or plates a species, while the second portion interacts with the species to a lesser extent. In another embodiment, fast portion 320 and second portion 322 may be formed of the same material. For example, first portion 320 and second portion 322 may be formed of the same material and may be substantially dense, or porous, such as a pressed or sintered powder or foam structure. In some cases, to produce a differential strain upon operation of the electrochemical cell, first portion 320 or second portion 322 may have sufficient thickness such that, during operation of the electrochemical cell, a gradient in composition may arise due to limited ion transport, producing a differential strain. In some embodiments, one portion or an area of one portion may be preferentially exposed to the species relative to the second portion or area of the second portion. In other instances, shielding or masking of one portion relative to the other portion can result in lesser or greater intercalation, de-intercalation, or alloying with the masked or shielded portion compared to the non-masked or shielded portion. This may be accomplished, for example, by a surface treatment or a deposited barrier layer, lamination with a barrier layer material, or chemically or thermally treating the surface of the portion to be masked/shielded to either facilitate or inhibit intercalation, de-intercalation, alloying, oxidation, reduction, or plating with the portion. Barrier layers can be formed of any suitable material, which may include polymers, metals, or ceramics. In some cases, the barrier layer can also serve another function in the electrochemical cell, such as being a current collector. The barrier layer may be uniformly deposited onto the surface in some embodiments. In other cases, the barrier layer may form a gradient in composition and/or dimension such that only certain portions of the surface preferentially facilitate or inhibit intercalation, de-intercalation, alloying, oxidation, reduction, or plating of the surface. Linear, step, exponential, and other gradients are possible. In some embodiments a variation in the porosity across first portion 320 or second portion 322, including the preparation of a dense surface layer, may be used to assist in the creation of an ion concentration gradient and differential strain. Other methods of interaction of a species with a first portion to a different extent so as to induce a differential strain between the first and second portions can also be used. In some embodiments, the flexure or bending of an electrode is used to exert a force or to carry out a displacement that accomplishes useful function.

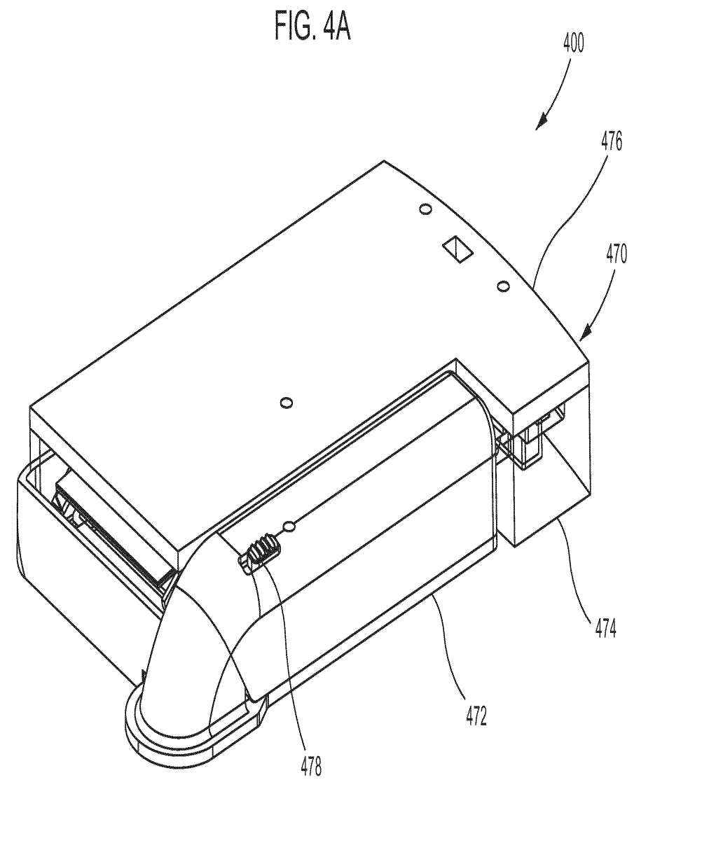

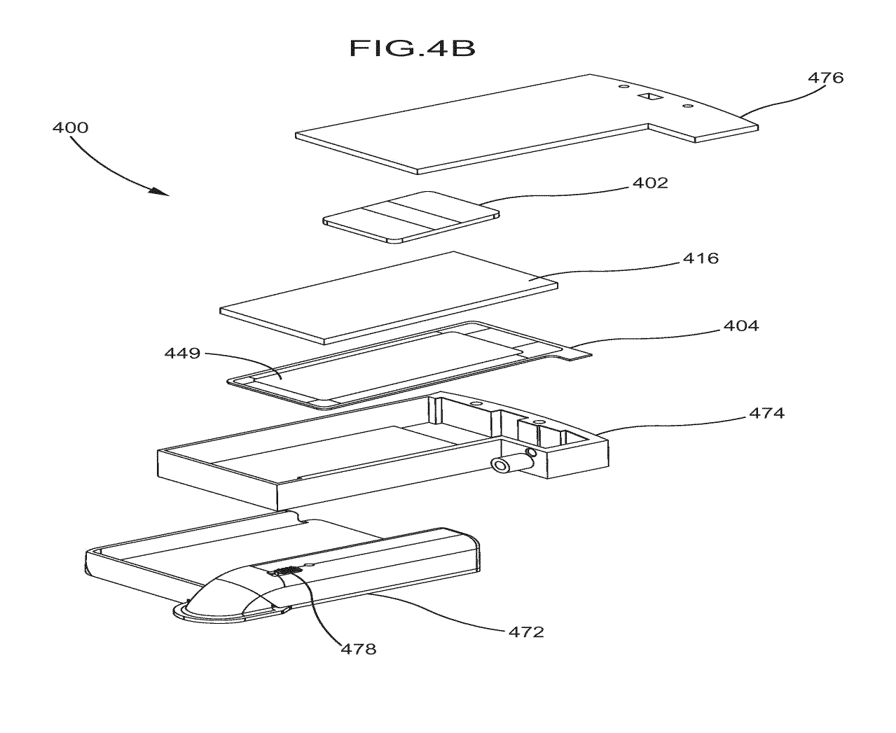

FIGS. 4A and 4B illustrate an embodiment of a delivery device that can include an electrochemical actuator as described herein. A delivery device 400 includes a housing 470, a fluid source 404, an electrochemical actuator 402, a transfer structure 416 can be disposed between the fluid source 404 and the actuator 402, and associated electronics (not shown) that can be coupled to the electrochemical actuator 402. In this embodiment, the housing 470 includes a first portion 472, a second portion 474, and a top portion 476 that can be coupled together to form an interior region within the housing 470. The fluid source 404, the electrochemical actuator 402 and the transfer structure 416 can each be disposed within the interior region defined by the housing 470.

The fluid source 404 can be provided to a user already disposed within the interior region of the housing 470 or can be provided as a separate component that the user can insert into the housing 470. For example, the fluid source 404 can be inserted through an opening (not shown) in the housing 470. The fluid source 404 can be, for example, a fluid reservoir, bag or Container, etc. that defines an interior volume that can contain a fluid to be injected into a patient. The fluid source 404 (also referred to herein as “fluid reservoir”) can include a web portion (not shown) configured to be punctured by an insertion mechanism (not shown) to create a fluid channel between the fluid source 404 and a fluid communicator (not shown) configured to penetrate the patient's skin. In some embodiments, the fluid reservoir 404 can be sized for example, with a length L of about 2 cm, a width W of about 2 cm, and a height H of about 0.25 cm, to contain, for example, a total volume of 1 ml of fluid.

The delivery device 400 also includes an activation mechanism 478 in the form of a button that can be used to activate the insertion mechanism and/or the actuator 402. The first portion 472, the second portion 474 and the top portion 476 of the housing 470 can be coupled together in a similar manner as with various embodiments of a delivery system described in the '771 Patent incorporated by reference above. The first portion 472, the second portion 474 and the top portion 476 can be coupled, for example, with an adhesive, a snap fit coupling or other known coupling method. The first portion 472 can be adhered to a patient's body with an adhesive layer disposed on a bottom surface of the first portion 472.

To use the delivery device 400, the delivery device 400 is placed at a desired injection site on a patient's body and adhesively attached thereto. When the fluid source 404 is disposed within the housing 470 (e.g., inserted into the housing by the patient or predisposed), the activation mechanism 478 (e.g., button, switch, lever, pull-tab, etc.) can be moved from an of position to an on position, which will cause the fluid communicator to penetrate the patient's skin at the treatment site. Alternatively, in some embodiments, the insertion mechanism (not shown) can be activated by the fluid source 404 being inserted into the housing.

The electrochemical actuator 402 can be activated after the insertion mechanism has been activated and the fluid communicator is inserted into the patient's body. Alternatively, in some embodiments, the electrochemical actuator 402 can be activated simultaneously with activation of the insertion mechanism. For example, when the insertion mechanism is activated it can be configured to activate a trigger mechanism (not shown) that communicates with the electrochemical actuator 402. For example, such a trigger mechanism can complete the electric circuit (as described above) and cause the electrochemical actuator 402 to start discharging. As the electrochemical actuator 402 discharges, the actuator 402 will displace and exert a force on the transfer structure 416, which in turn will exert a force on the top surface 449 of the fluid source 404, thereby compressing the fluid source 404 between the transfer structure 416 and the second portion 474 of the housing 470 and causing a volume of fluid within the fluid source 404 to be expelled into the patient.

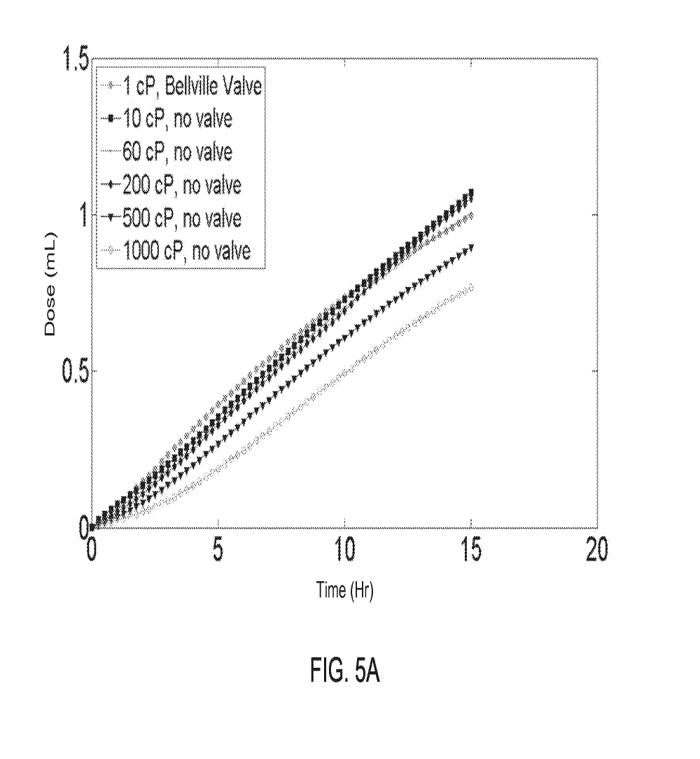

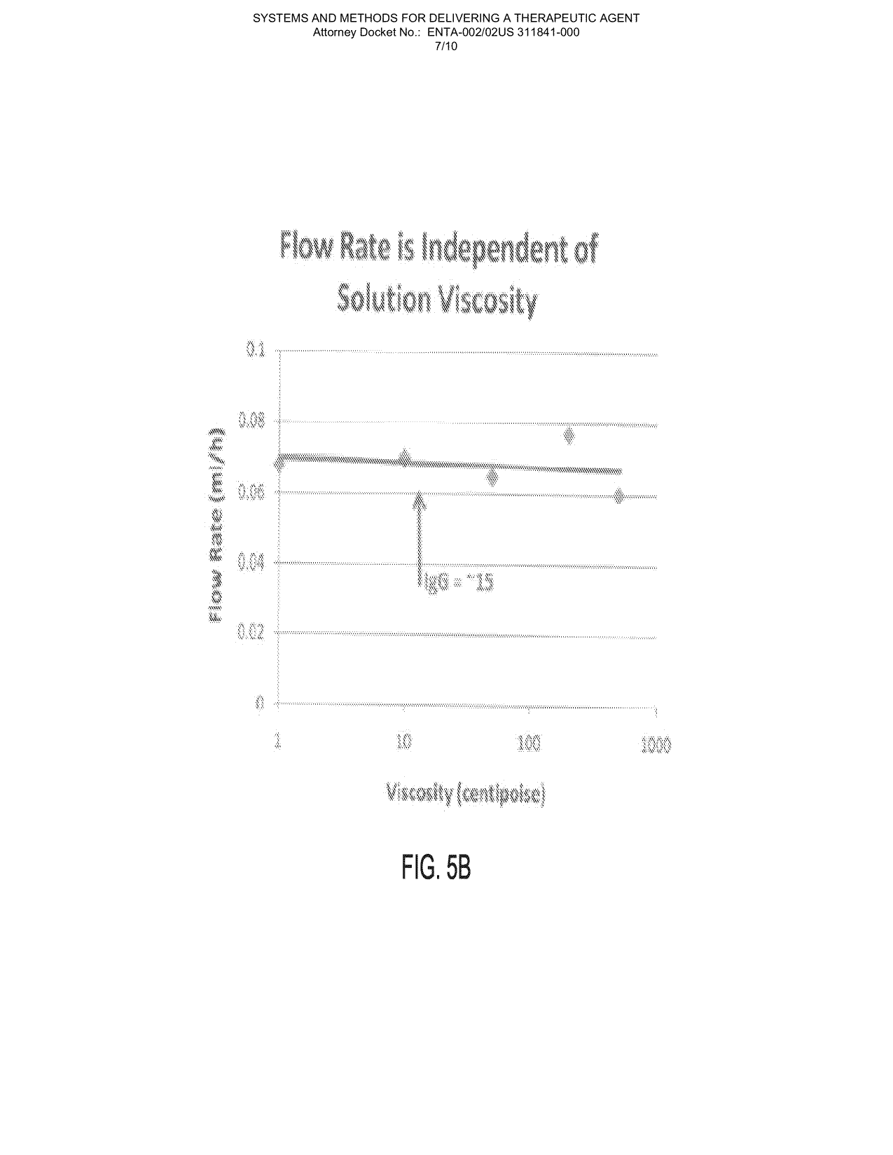

A patch pump device as described herein can be used to pump highly viscous fluids, such as, for example, fluids with viscosity greater than 15 cP. The unique capabilities of an electrochemical actuator enable pumps based on this type of actuator to pump unusually high viscosity fluids, such as those including biologics, with little to no loss in pumping rate. FIG. 5A is a graph of example doses versus time that can be delivered using a patch pump device with such an electrochemical actuator; and FIG. 5B is a graph of example flow rates versus viscosity. As shown in FIG. 5B, the average delivery rate (i.e., flow rate) is substantially unaffected by viscosity below 500 cP. A patch pump device as described herein, is capable, of pumping fluids with viscosity up to approximately 2,000 cP or higher. For example, in some embodiments, the patch pump device is capable of pumping fluids with viscosity in a range of about 0.5 cP to about 2,000 cP. In some embodiments, the patch pump device is capable of pumping fluids with viscosity in any sub-range within this range. For example, in some embodiments, the patch pump device is capable of pumping fluids with viscosity in a range of about 1 cP to about 1,000 cP. In some embodiments, the patch pump device is capable of pumping fluids with viscosity in a range of about 10 cP to about 500 cP.

Thus, an electrochemical actuator as described herein can deliver fluids having viscosities similar to many biologics at reasonable delivery rates. Pumps based on a solid-state transformation based mechanism tend to be able to exert very large forces; and as the viscosity of the pumping fluid increases, the force required to pump the liquid increases correspondingly.

The relationship between force and flow is dependent on the type of flow and the shape of the pipe (e.g., needle) where the flow is occurring. Since needles typically have a round cross section and since the flow rate through a needle during an injection is relatively low, the flow can be determined with reasonable accuracy by using Poiseuille's equation for laminar flow in a pipe. This equation relates the pressure drop, ΔP, required to cause fluid of viscosity, μ, to flow at a flow rate Q through a pipe of length L and radius r.

ΔP=(8μLQ)/(πr⁴)

The required force to cause fluid to flow can be calculated by multiplying the pressure drop by the area over which the force will act. For a syringe, force is applied to a plunger (which is essentially a piston in the cylindrical barrel of the syringe) to pressurize the fluid contained in the barrel and produce the pressure drop between the barrel outlet (needle inlet) and the distal end of the needle. Thus, the pressure in the barrel is simply the force on the plunger divided by the area of the plunger. Of course, if only the plunger is pushed, the entire syringe would move, so the barrel must be constrained by application of an equal and opposite force.

For a patch pump device using an electrochemical actuator as described herein, the drug reservoir can be, for example, a bag which is compressed between two surfaces, e.g. plates. The force required to be applied to each side of the bag to generate the desired pressure drop across the outlet tube is simply the pressure times the contact area between each plate and the bag.

F=A_{c}ΔP=A_{c}(8μLQ)/(πr⁴)

Where F is the force and A_{c} is the contact area of the bag to which the force is applied.

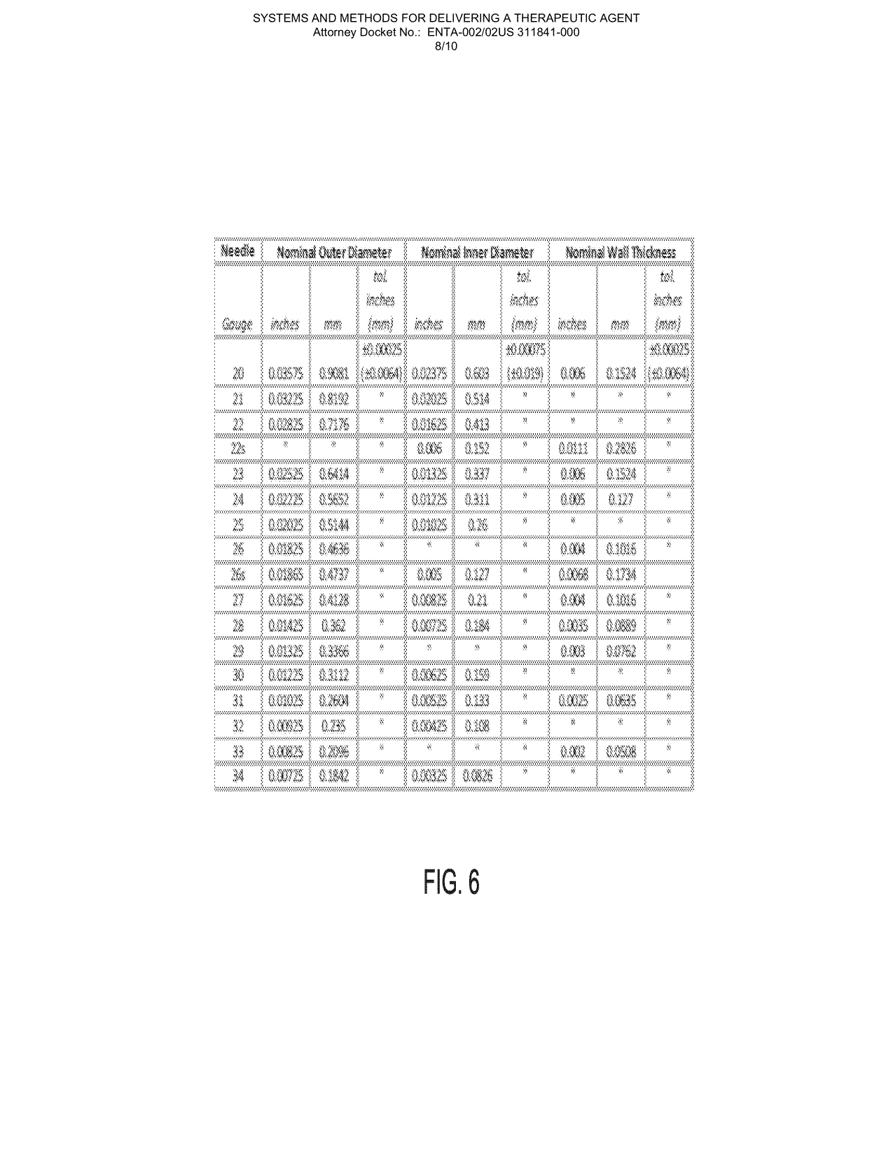

Most fluids for injection are relatively inviscid, having viscosities near that of water (1 cP). However, some formulations can have higher viscosities up to, for example, about 15 cP. Rarely will fluids for injection be formulated at higher viscosity than 15 cP because the force required to push them through a needle of reasonably small size using a standard syringe becomes too large. A standard 1 ml syringe has an inner barrel diameter of, for example, 0.47 cm and a 30 gauge needle can have a diameter as listed in the table shown in FIG. 6 and a length of about 2 cm (about 0.5 inches). Using the equation above, it can be determined that it requires nearly 2 pounds of force to dispense 1 ml of water in 5 seconds from a 1 ml syringe through a 30 gauge needle as shown in Table 1 below. The required force scales directly with the viscosity of the fluid and quickly becomes quite large. An electrochemical actuator as described herein, is capable of delivering a force up to approximately 500 pounds of force or higher. For example, in some embodiments, the electrochemical actuator is capable of delivering a force in a range of about 1 pound to about 500 pounds. In some embodiments, the electrochemical actuator is capable of delivering a force in any sub-range within this range. For example, in some embodiments, the electrochemical actuator is capable a delivering a force in a range of about 2 pounds to about 250 pounds. In some embodiments, the electrochemical actuator is capable of delivering a force in a range of about 5 pounds to about 200 pounds.

TABLE 1
Force required to inject 1 ml in 5 seconds
using a 1 ml syringe and a 30 gauge needle

| | 1 cP | 15 cP | 50 cP |
|---|---|---|---|
| Newtons | 8.848042 | 132.7206 | 442.4021 |
| Pounds | 1.98904 | 29.8356 | 99.45199 |

The ability to deliver viscous fluids (e.g., fluids with viscosity greater than about 15 cP) using a patch pump including an electrochemical actuator is due in part to the fact that the patch pump may be worn for extended periods of time, therefore allowing the delivery of the fluid to extend over a longer period of time. This results in a much slower required flow rate through the needle, and thus a smaller required force. However, because the patch pump is worn by the user, the aspect ratio of the delivery system changes such that the a syringe system is not appropriate—the barrel of the syringe becomes larger than a height and length of the system that is convenient for wearing.

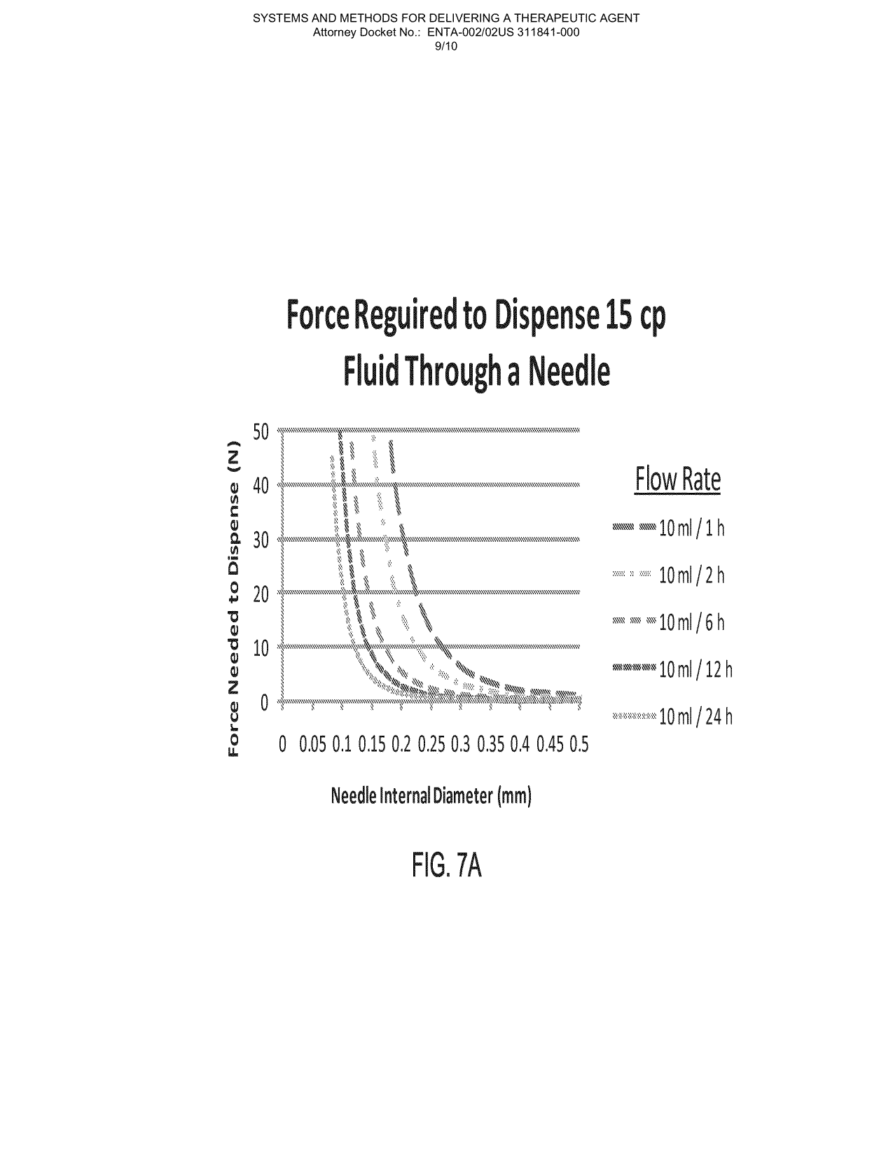

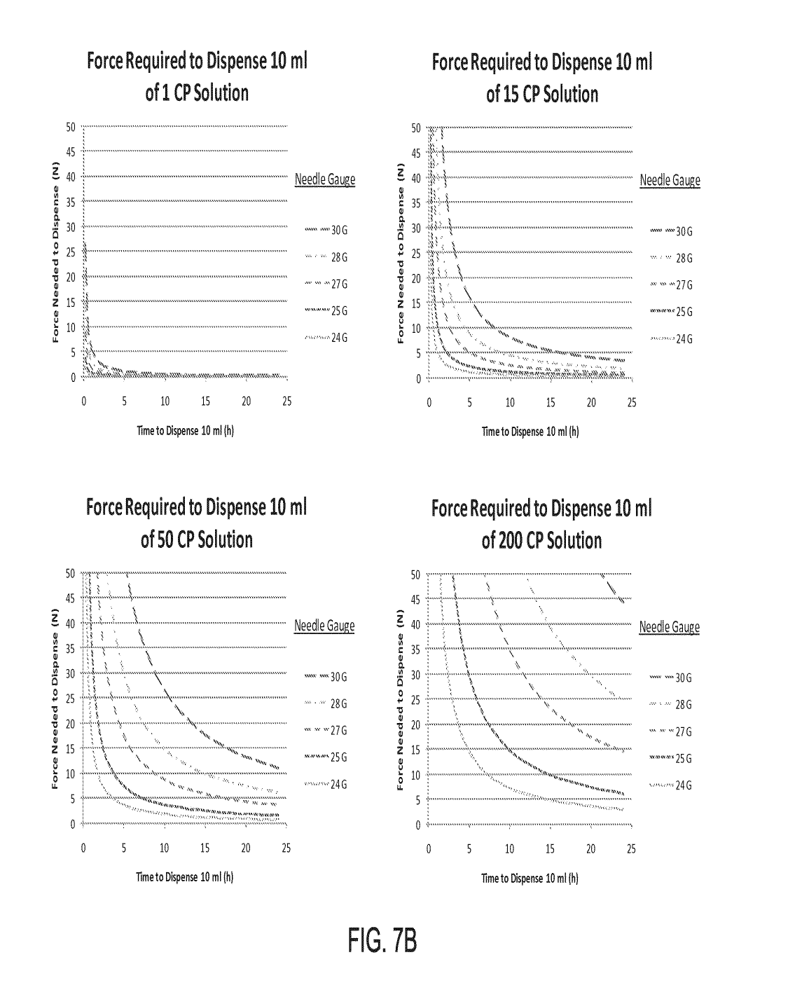

A number of advantages exist for an actuator that is capable of exerting large force. For example, the volume delivered can be increased substantially. Since increasing the volume delivered requires the flow rate to be increased, this also results in a larger required force. In another example, an actuator capable of exerting a large force can deliver fluids having higher viscosities. FIG. 7A shows examples of the calculated effect of viscosity on the required force needed to pump a fluid volume of 10 ml at various rates, and also the interrelationship between the diameter of the tube the fluid is being pumped through (the needle gauge) and the desired rate at which the fluid is being pumped. FIG. 7B shows graphs showing examples of the force required to dispense various amounts of fluids having various viscosities over time.

Current pharmaceutical formulations of injectable products are limited by the need to keep viscosity at an acceptably low level (e.g. about 15 cp or less). With the ability to pump high viscosity fluids, the use of new formulations that have much higher viscosities will be possible. For example, some pharmaceutical compounds/formulations that may be particularly benefited by allowing high viscosity formulations include biologics, which tend to have dramatically increased viscosity as their concentration increases above about 50 mg/ml. For example, a pump device as described herein, is capable of delivering biologics having concentrations in a range of about 10 mg/ml to about 1 g/ml. In some embodiments, the pump device is capable of delivering biologics having concentrations in any sub-range within this range. For example, in some embodiments, the pump device is capable of delivering biologics having concentrations in a range of about 50 mg/ml to about 500 mg/ml. In some embodiments, the pump device is capable of delivering biologics having concentrations in a range of about 100 mg/ml to about 250 mg/ml. Other pharmaceutical compounds/formulations that may be particularly benefited by allowing high viscosity formulations include 1) formulation and stability additives that tend to increase viscosity, such as small molecular weight PEG solutions at high concentration or large molecular weight PEG solutions at moderate concentrations, glycerine, pharmaceutically acceptable sugars, and other polymeric additive—HEC, HPMC, pluronics, etc., 2) polymeric and solid solution based depot formulations, 3) nucleic acids, such as aptamers, antisense, RNAi and siRNA, and 4) pegylated polypeptides.

In addition, pump devices using electrochemical actuators may provide a more uniform delivery of depot formulations which has the added benefit of creating a more uniform depot “slug” of material. This can make both the burst phase and the delivery phase of the depot formulation more uniform from patient to patient and from injection to injection within the same patient.

In some embodiments, the fluid source 104 is a flexible fluid reservoir, such as a bag, and the fluid reservoir is squeezed between two surfaces, such as between a piston (or other transfer structure) and a surface of the patch pump housing. In some embodiments, the bag is squeezed between an electrochemical actuator and a surface of the patch pump housing. In some embodiments, such a fluid reservoir can be sized for example, with a length L of about 2 cm, a width W of about 2 cm, and a height H of about 0.25 cm, to contain, for example, total volume of 1 ml of fluid. Such a reservoir can be pressurized by applying a force to a contact area A_{cr} in the height direction of the fluid reservoir of

A_{cr}=L×W=(2×2)cm²=4 cm².

For a typical syringe with a barrel diameter of 0.47 cm the contact area A_{cs} can be calculated as follows:

A_{cs}=πd²/4=π(0.47)²/4=0.1735 cm²

The ratio of the contact area of a fluid reservoir as described above and the Contact area of a typical syringe is:

A_{cr}/A_{cs}=4 cm²/0.1735 cm²=23.1

This 23 fold increase in contact area for the fluid reservoir as compared to the syringe correspondingly requires a 23 fold increase in injection force to generate the same fluid pressure. However, by slowing down the injection time from, for example, 5 seconds to 2 minutes, a 24 fold decrease, the required injection force for a patch pump can be the same as the injection force for a 1 ml syringe. A further lengthening of the delivery time beyond 2 minutes can allow the patch pump to deliver 1 ml of fluid with lower overall force than would be required using a 1 ml syringe.

One example of a delivery device 100 is configured to deliver 5 ml over a 4 hour time period at a uniform delivery rate of 1.2 ml/hour. The fluid source 104 is a 4 cm×4 cm×0.3 cm thick bag and the fluid communicator 106 is a 3.8 cm long, 27 gauge needle. The force required to deliver the 5 ml over the 4 hour time period is 0.10 pounds for a 1 cP solution, 1.43 pounds for a 15 cP solution, and 4.77 pounds for a 50 cP solution.

Another example of a delivery device 100 is also configured to deliver 5 ml over a 4 hour time period at a uniform delivery rate of 1.2 ml/hour. The fluid source 104 is also a 4 cm×4 cm×0.3 cm thick bag but the fluid communicator 106 is a 3.8 cm long, 30 gauge needle. The force required to delivery the 5 ml over the 4 hour time period is 0.29 pounds for a 1 cP solution, 4.36 pounds for a 15 cP solution, and 14.52 pounds for a 50 cP solution.

Another example of a delivery device 100 is configured to deliver 10 ml over a 6 hour time period at a uniform delivery rate of 1.667 ml/hour. The fluid source 104 is a 5 cm×5 cm×0.43 cm thick bag and the fluid communicator 106 is a 3.8 cm long, 27 gauge needle. The force required to delivery the 10 ml over the 6 hour time period is 0.21 pounds for a 1 cP solution, 3.11 pounds for a 15 cP solution, and 10.36 pounds for a 50 cP solution.

Another example of a delivery device 100 is also configured to deliver 10 ml over a 6 hour time period at a uniform delivery rate of 1.667 ml/hour. The fluid source 104 is also a 5 cm×5 cm×0.43 cm thick bag but the fluid communicator 106 is a 3.8 cm long, 30 gauge needle. The force required to delivery the 10 ml over the 6 hour time period is 0.63 pounds for a 1 cP solution, 9.46 pounds for a 15 cP solution, and 31.51 pounds for a 50 cP solution.

As shown in Table 2 below, delivery device 100 can be volumetrically efficient, meaning that the volume of the payload delivered by the device is a relatively high percentage of the overall volume of the device. This volumetric efficiency can increase with the volume of the payload, i.e. an increase in payload volume does not require a proportional increase in the total volume of the delivery device. As described above, the delivery device 100 can include a housing that comprised of a single or multiple components. In certain embodiments, the cannula insertion mechanism can be integrated into the housing with the fluid source. In other embodiments, the cannula insertion mechanism can be a separate component, thus increasing the volumetric efficiency of the delivery device 100.

TABLE 2
Fluid Source Volume as a Percentage of Delivery Device Volume

| | Percent of Total | Percent of Total |
|---|---|---|
| Fluid Source | Volume with Integrated | Volume without Integrated |
| Volume (ml) | Insertion Mechanism | Insertion Mechanism |
| 2 | >10% | >45% |
| 5 | >20% | >45% |
| 10 | >25% | >45% |
| 20 | >30% | >45% |

A fluid delivery device as described herein may be used to deliver a variety of drugs according to one or more release profiles. For example, the drug may be delivered according to a relatively uniform flow rate, a varied flow rate, a preprogrammed flow rate, a modulated flow rate, in response to conditions sensed by the device, in response to a request or other input from a user or other external source, or combinations thereof. Thus, embodiments of the delivery device may be used to deliver drugs having a short half-life, drugs having a narrow therapeutic window, drugs delivered via on-demand dosing, normally-injected compounds for which other delivery modes such as continuous delivery are desired, drugs requiring titration and precise control, and drugs whose therapeutic effectiveness is improved through modulation delivery or delivery at a non-uniform flow rate. These drugs may already have appropriate existing injectable formulations.

A particular benefit of the delivery devices of the current invention is the avoidance of toxicity resulting from high maximum blood concentrations (Cₘₐₓ) that are often associated with high dose intravenous and/or subcutaneous transfusions. The delivery devices of the current invention, by delivering therapeutics over a prolonged period of time, effectively improve efficacy with a higher minimum blood concentration (Cₘᵢₙ) and thereby avoids toxicity associated with high Cₘₐₓ.

For example, the delivery devices may be useful in a wide variety of therapies. Representative examples include, but are not limited to, opioid narcotics such as fentanyl, remifentanyl, sufentanil, morphine, hydromorphone, oxycodone and salts thereof or other opioids or non-opioids for post-operative pain or for chronic and breakthrough pain; NonSteroidal Antinflamatories (NSAIDs) such as diclofenac, naproxen, ibuprofin, and celecoxib; local anesthetics such as lidocaine, tetracaine and bupivicaine; dopamine antagonists such as apomorphine, rotigotine, and ropinerole; drugs used for the treatment and/or prevention of allergies such as antihistamines, antileukotrienes, anticholinergics, and immunotherapeutic agents; antispastics such as tizanidine and baclofin; insulin delivery for Type 1 or Type 2 diabetes; leutenizing hormone releasing hormone (LHRH) or follicle stimulating hormone (FSH) for infertility; plasma-derived or recombinant immune globulin or its constituents for the treatment of immunodeficiency (including primary immunodeficiency), autoimmune disorders, neurological and neurodegenerative disorders (including Alzheimer's Disease), and inflammatory diseases; apomorphine or other dopamine agonists for Parkinson's disease; interferon A for chronic hepatitis B, chronic hepatitis C, solid or hematologic malignancies; antibodies for the treatment of cancer; octreotide for acromegaly; ketamine for pain, refractory depression, or neuropathic pain; heparin for post-surgical blood thinning; corticosteroid (e.g., prednisone, hydrocortisone, dexamethasone) for treatment of MS; vitamins such as niacin; Selegiline; and rasagiline. Essentially any peptide, protein, biologic, or oligonucleotide, among others, that is normally delivered by subcutaneous, intramuscular, or intravenous injection or other parenteral routes, may be delivered using embodiments of the devices described herein. In some embodiments, the delivery device can be used to administer a drug combination of two or more different drugs using a single or multiple delivery port and being able to deliver the agents at a fixed ratio or by means enabling the delivery of each agent to be independently modulated. For example, two or more drugs can be administered simultaneously or serially, or a combination (e.g. overlapping) thereof.

In some embodiments, the delivery device may be used to administer ketamine for the treatment of refractory depression or other mood disorders. In some embodiments, ketamine may include either the racemate, single enantiomer (R/S), or the metabolite (wherein S-norketamine may be active). In some embodiments, the delivery devices described herein may be used for administration of Interferon A for the treatment of hepatitis C. In one embodiment, a several hour infusion patch is worn during the day or overnight three times per week, or a continuous delivery system is worn 24 hours per day. Such a delivery device may advantageously replace bolus injection with a slow infusion, reducing side effects and allowing the patient to tolerate higher doses. In other Interferon A therapies, the delivery device may also be used an the treatment of malignant melanoma, renal cell carcinoma, hairy cell leukemia, chronic hepatitis B, condylomata acuminata, follicular (non-Hodgkin's lymphoma, and AIDS-related Kaposei's sarcoma.

In some embodiments, a delivery device as described herein may be used for administration of apomorphine or other dopamine agonists in the treatment of Parkinson's Disease (“PD”). Currently, a bolus subcutaneous injection of apomorphine may be used to quickly jolt a PD patient out of an “off” state. However, apomorphine has a relatively short half-life and relatively severe side effects, limiting its use. The delivery devices described herein may provide continuous delivery and may dramatically reduce side effects associated with both apomorphine and dopamine fluctuation. In some embodiments, a delivery device as described herein can provide continuous delivery of apomorphine or other dopamine agonist, with, optionally, an adjustable baseline and/or a bolus button for treating an “off” state in the patient. Advantageously, this method of treatment may provide improved dopaminergic levels in the body, such as fewer dyskinetic events, fewer “off” states, less total time in “off” states, less cycling between on and “off” states, and reduced need for levodopa; quick recovery from “off” state if it occurs; and reduced or eliminated nausea/vomiting side effect of apomorphine, resulting from slow steady infusion rather than bolus dosing.

In some embodiments, a delivery device as described herein may be used for administration of an analgesic, such as morphine, hydromorphone, fentanyl or other opioids, in the treatment of pain. Advantageously, the delivery device may provide improved comfort in a less cumbersome and/or less invasive technique, such as for post-operative pain management. Particularly, the delivery device may be configured for patient-controlled analgesia.

### CONCLUSION

While various embodiments of the invention have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and that such modifications are in accordance with the variations of the invention. Additionally, certain of the steps may be performed concurrently in a parallel process when possible, as well as performed sequentially as described above. The embodiments have been particularly shown and described, but it will be understood various changes in form and details may be made.

For example, although various embodiments have been described as having particular features and/or combinations of components, other embodiments are possible having any combination or sub-combination of any features and/or components from any of the embodiments described herein. For example, although some embodiments were not described as including an insertion mechanism, an activation mechanism, electrical circuitry, etc., it should be understood that those embodiments of a delivery device can include any of the features, components and/or functions descried herein for other embodiments. In addition, the specific configurations of the various components can also be varied. For example, the size and specific shape of the various components can be different than the embodiments shown, while still providing the functions as described herein.

What is claimed is:

1. A method of delivering a fluid with viscosity of greater than 15 cP, comprising:
actuating an electrochemical actuator such that the electrochemical actuator deflects, thereby exerting a force in the range of about 1 pound to about 500 pounds on a fluid reservoir contained within a wearable delivery device to cause the fluid within the reservoir to be communicated to the patient's body over a time period.

2. The method of claim 1, wherein the time period is in the range of about 15 minutes to about 7 days.

3. The method of claim 1, wherein the time period is in the range of about 30 minutes to about 72 hours.

4. The method of claim 1, wherein the time period is in the range of about 30 minutes to about 24 hours.

5. The method of claim 1, wherein the force exerted by the electrochemical actuator in the range of about 2 pounds to about 250 pounds.

6. The method of claim 1, wherein the force exerted by the electrochemical actuator in the range of about 5 pounds to about 200 pounds.

7. An apparatus, comprising:
a reservoir containing a fluid having a viscosity greater than 0.5 cP;
a fluid communicator in fluid communication with the reservoir;
an electrochemical actuator coupled to the reservoir and configured to exert a sufficient force on the reservoir for a time period upon actuation such that the fluid within the reservoir is communicated through the fluid communicator.

8. The apparatus of claim 7, further comprising:
a transfer structure disposed between the actuator and the reservoir, the transfer structure configured to contact the reservoir and transfer the force exerted by the electrochemical actuator to the reservoir upon actuation of the electrochemical actuator.

9. The apparatus of claim 7, wherein the reservoir and the electrochemical actuator are disposed within a housing, the housing configured to be removably attached to a patient's body.

10. The apparatus of claim 7, wherein the fluid has a viscosity in the range of about 0.5 cP to about 2,000 cP.

11. The apparatus of claim 7, wherein the fluid has a viscosity in the range of about 1 cP to about 1,000 cP.

12. The apparatus of claim 7, wherein the fluid has a viscosity in the range of about 1 cP to about 500 cP.

13. The apparatus of claim 7, wherein the reservoir contains about 0.1 ml to about 1,000 ml of fluid.

14. The apparatus of claim 7, wherein the reservoir contains about 0.5 ml to about 100 ml of fluid.

15. The apparatus of claim 7, wherein the reservoir contains about 0.5 ml to about 20 ml of fluid.

16. A patch pump device for delivering fluid to the body of a patient having a delivery device volume, the patch pump device comprising:
a reservoir containing a volume fluid having a viscosity greater than 0.5 cP, the volume of fluid being at least 10% of the delivery device volume;
a fluid communicator having a first configuration in which the fluid communicator is fluidically isolated from the reservoir and a second configuration in which the fluid communicator is in fluid communication with the reservoir;
an insertion mechanism operable to move the fluid communicator from its first configuration to its second configuration; and
an electrochemical actuator including an electrode configured to deflect as the electrochemical actuator discharged, the deflection of the electrode being operative to exert a sufficient force on the reservoir for a time period upon actuation such that the volume of fluid within the reservoir is communicated through the fluid communicator.

17. The patch pump device of claim 16, wherein the volume of fluid is at least 20% of the delivery device volume.

18. The patch pump device of claim 16, wherein the volume of fluid is at least 25% of the delivery device volume.

19. The patch pump device of claim 16, wherein the volume of fluid is at least 30% of the delivery device volume.

20. The apparatus of claim 7, wherein the volume of fluid is about 0.5 ml to about 20 ml of fluid.
